(19) United States
(12) Patent Application Publication
(10) Pub. No.: US 2013/0331389 A1
(43) Pub. Date: Dec. 12, 2013

(54) Methods and Compositions for Cardiomyocyte Replenishment by Endogenous and Progenitor Stem Cells

(71) Applicant: NATIONAL CHENG KUNG UNIVERSITY, TAINAN CITY (TW)

(72) Inventors: Ching Ho Hsieh, Tainan City (TW); Ying-Chang Hsueh, Kaohsiung City (TW); Jasmine M.F. Wu, Kaohsiung City (TW)

(73) Assignee: NATIONAL CHENG KUNG UNIVERSITY, TAINAN CITY (TW)

(21) Appl. No.: 13/795,405

(22) Filed: Mar. 12, 2013

Related U.S. Application Data
(60) Provisional application No. 61/657,966, filed on Jun. 11, 2012.

(51) Int. Cl. A61K 31/5575; A61K 31/4709; A61K 31/444; A61K 31/498; A61K 31/5377; A61K 31/4439; A61K 31/519

(52) U.S. Cl. CPC A61K 31/5575; A61K 31/4439; A61K 31/4709; A61K 31/519; A61K 31/498; A61K 31/5377; A61K 31/444
USPC 514/233.2; 514/338; 514/314; 514/252.16; 514/249; 514/333; 514/300; 514/573

(57) ABSTRACT

Disclosed herein are methods and compositions for replenishing injured and/or damaged cardiomyocytes in a subject by inducing, increasing, and/or enhancing the differentiation of endogenous stem and progenitor cells in the subject.

### CROSS-REFERENCE TO RELATED APPLICATIONS

This application claims the benefit of U.S. Application Ser. No. 61/657,966, filed 11 Jun. 2012, which is herein incorporated by reference in its entirety.

### REFERENCE TO A SEQUENCE LISTING SUBMITTED VIA EFS-WEB

The content of the ASCII text file of the sequence listing named “20130312_{—}034535_{—}002 seq_ST25” which is 9.14 kb in size was created on 11 Mar. 2013 and electronically submitted via EFS-Web herewith the application is incorporated herein by reference in its entirety.

### BACKGROUND OF THE INVENTION

1. Field of the Invention

The present invention generally relates to methods and compositions for treating damaged and/or diseased cardiac tissue in subjects.

2. Description of the Related Art

It has long been thought that the adult mammalian heart is a mitotically quiescent organ with a limited regenerative ability. However, growing studies have demonstrated that the adult mammalian heart preserves a self-renewal capacity (Hsieh, P. C. H. et al. Nat. Med. 13, 970-974 (2007) and Senyo, S. E. et al. Nature 493, 433-436 (2013)) and various stem/progenitor cell populations residing in the heart have also been identified (Hoch, M. et al. Cell Stem Cell 9, 131-143 (2011); Laugwitz, K.-L. et al. Nature 433, 647-653 (2005); Oh, H. et al. Proc. Natl. Acad. Sci. USA 100, 12313-12318 (2003); Rota, M. et al. Proc. Natl. Acad. Sci. USA 104, 17783-17788 (2007); Pfister, O. et al. Circ. Res. 97, 52-61 (2005); and Smith, R. R. et al. Circulation 115, 896-908 (2007)). Further evidence indicates that exogenous stimuli may improve the regenerative capability of young murine heart (Smart, N. et al. Nature 474, 640-644 (2011); and Loffredo, Francesco S., et al. Cell Stem Cell 8, 389-398 (2011)). Interestingly, a recent study showed that following myocardial infarction (MI), epicardial Wilms tumor 1⁺ (Wt1) progenitor cells migrate to the injured region and differentiate into cardiomyocytes, which can be further improved by administration of thymosin β4 before MI (Smart (2011)).

Unfortunately, the prior art teaches that pre-existing cardiomyocytes are the primary cells that are lost and/or damaged after an injury to the heart and that the stem cells and progenitor cells have a very limited role in cardiomyocyte replacement (Senyo, S. E. et al. Nature 493, 433-436 (2013)) as after 8 weeks post-infarction, only about 3.2% of newly generated cardiomyocytes with the result of endogenous stem cells and/or progenitor cells or self-duplication from cardiomyocytes themselves.

### SUMMARY OF THE INVENTION

In some embodiments, the present invention provides methods for replenishing lost and/or damaged cardiomyocytes in a cardiac tissue in subjects which comprise inducing, increasing, and/or enhancing endogenous stem cells and/or progenitor cells in the subject to differentiate into new cardiomyocytes by a) binding and/or activating the prostaglandin E2 receptors; and/or b) attenuating and/or inhibiting the TGF-β1 signaling pathway; in the subjects. In some embodiments, the attenuating and/or inhibiting the TGF-β1 signaling pathway is by administering to the subjects at least one TGF-β1 signaling inhibitor such as SB 431542, LY2157299, LDN193189, SB 525334, LY2109761, SB505124, GW788388, Pirfenidone, Y364947, IDT-1, E-616452, and E-616451, preferably IDT-1, E-616452,451, SB 525334, or LY-364947. In some embodiments, the TGF-β1 signaling inhibitor is one as described in U.S. Pat. Nos. 5,958,411; 6,329,500; 6,500,920; 6,673,341; 7,173,002; 7,420,050; 7,407,958; or 8,110,655. In some embodiments, the binding and/or activating the prostaglandin E2 receptors is by administering to the subjects an effective amount of a PGE2 compound and/or a PGE2 agonist. In some embodiments, the PGE2 compound is prostaglandin E2 or a salt thereof, a derivative of a prostaglandin receptor, or a compound having as part of its structural backbone, the following structural formula (I), which may or may not be substituted:

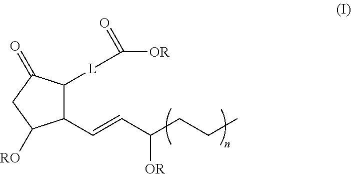

wherein n is an integer between 1 and 10, preferably between 1 and 5, more preferably 2; L is a linker which can be a substituted or unsubstituted alkyl, a substituted or unsubstituted alkenyl; and the R's are each independently H, a halogen, a substituted or unsubstituted C₁₋₆ alkyl, or a substituted or unsubstituted C₁₋₆ alkenyl; and acid, bases, and salts thereof. In some embodiments, the PGE2 compound is a compound as disclosed in U.S. Pat. Nos. 5,663,417; 6,046,236; 6,426,359; 6,531,485; 7,053,085; 7,238,710; 7,326,732; 7,109,223; 7,238,710; or 8,063,240. In some embodiments, the PGE2 compound and/or the PGE2 agonist is selected from the group consisting of PGE2 and salts thereof, analogues and derivatives of PGE2 and salts thereof, butaprost, CP-533,536, ONO-AE1-259-01, sulprostone, enprostil, and ONO-4819. In some embodiments, the a) binding and/or activating the prostaglandin E2 receptors; and/or b) attenuating and/or inhibiting the TGF-β1 signaling pathway is conducted before, during and/or after an event likely to cause injury and/or damage to the cardiac tissue. In some embodiments, the a) binding and/or activating the prostaglandin E2 receptors; and/or b) attenuating and/or inhibiting the TGF-β1 signaling pathway is conducted up to about 3 months, preferably up to about 30 days, more preferably up to about 7-10 days after the event. In some embodiments, the cardiac tissue is injured or damaged by a myocardial infarction (MI), ischemia, hypoxia, coronary artery disease (CAD), cardiomyopathy, atherosclerosis, heart failure, congenital heart diseases, valvular heart diseases, ischemic heart diseases, and other conditions which damage the cardiac tissue such as viral infection or trauma. In some embodiments, the cardiovascular disease is heart disease, coronary artery disease, cardiomyopathy, myocardial infarction, atherosclerosis, heart failure, a congenital heart disease, a valvular heart disease, or an ischemic heart disease. In some embodiments, the effective amount of the PGE2 compound is administered orally, nasally, dermally, mucosally, intravenously, subcutaneously, intramuscularly, or intraperitoneally. In some embodiments, the effective amount of the PGE2 compound is about 0.001 mg/kg to about 0.1 mg/kg body weight, preferably about 0.001-0.01 mg/kg body weight, or more preferably about 0.0015-0.003 mg/kg body weight, of the subject. In some embodiments, the effective amount of the PGE2 compound is administered in conjunction with one or more cells, e.g., stem cells, progenitor cells, cardiomyocytes, etc., to be transplanted to the subject.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIGS. 1A-1B evidence that cardiomyocyte replenishment saturates on day 10 after heart injury. The adult cardiomyocytes in the MerCreMer/ZEG (M/Z) mice were labeled with GFP following 14 days of tamoxifen (TAM) injection, while the other cell types, including cardiac stem/progenitor cells, remained β-Gal⁺. M/Z mice treated with TAM underwent coronary artery ligation to induce myocardial infarction (MI). The hearts were isolated at different time points after MI and stained for GFP or β-Gal to examine the degree of cardiomyocyte replenishment. FIG. 1A are bar graphs summarizing the quantification of GFP⁺ and β-Gal⁺ cardiomyocytes at the border zone of injured heart at different time points. FIG. 1B are bar graphs summarizing the quantification of GFP⁺ and β-Gal⁺ cardiomyocytes at the remote area of injured heart at different time points. These results demonstrate that as early as 10 days after myocardial infarction, endogenous stem/progenitor cell-derived cardiomyocyte replenishment saturates.

FIGS. 2A-2B evidence that cardiomyocyte proliferation and progenitor cell activity are stimulated at early time points following injury. FIG. 2A are bar graphs presenting the percentage of β-Gal⁺/GFP⁻/Nkx2.5⁺ and β-Gal⁻/GFP⁺/NKx2.5⁺ cardiomyocytes per total (3-Gal⁺/GFP⁻ and β-Gal⁻/GFP⁺ cells, respectively. The hearts of tamoxifen-pulsed MerCreMer/ZEG (M/Z) mice underwent surgery-induced myocardial infarction (MI) were collected at different time points. The number of cardiomyocytes co-expressing cardiac progenitor cell marker Nkx2.5 was quantified. n≧3. FIG. 2B are graphs providing the percentages of non-myocytes or the myocytes labeled by BrdU in total non-myocytes or myocytes, respectively. The MI hearts were harvested to examine the degree of cardiomyocyte and non-myocyte undergoing active cell cycle, labeled by BrdU, at different time points after injury. The BrdU⁺ cells at the border and remote regions were quantified and statistically analyzed. n≧3. Data are presented as the mean±s.e.m. These results demonstrate that within 14 days post-MI, the majority of new cardiomyocyte formation are derived from endogenous stem/progenitor cells with some from self-duplication.

FIGS. 3A-3F evidence that the early inflammatory response modulated by PGE2 regulates cardiomyocyte replenishment post-infarction. After 14 daily tamoxifen injections, the hearts were allowed to recover for 1 month then induced with myocardial infarction (MI). Anti-inflammatory drugs were given at different time points, including indomethacin (Indo) and Celecoxib treatments 1 day before and 14 days (Indo 14 D and Celecoxib) or 5 days (Indo 5 D) after MI or Indo given from day 5 to day 14 post-MI (Indo last 9 days or Indo 9 D). FIG. 3A provides graphs of the percentages of GFP⁺ and β-Gal⁺ cardiomyocytes observed at the border zone that were quantified and statistically analyzed. FIG. 3B provides graphs of the percentages of GFP⁺ and β-Gal⁺ cardiomyocytes observed at the remote area that were quantified and statistically analyzed. After surgery-induced infarction, indomethacin, PGI2 or PGE2 were administered continuously for 14 days. FIG. 3C provides graphs of the percentages of GFP⁺ and β-Gal⁺ cardiomyocytes detected at the border zone that were quantified and statistically analyzed following PGE2 and/or indomethacin or PGI₂ treatments. FIG. 3D provides graphs of the percentages of GFP⁺ and β-Gal⁺ cardiomyocytes detected at the remote area were quantified and statistically analyzed. FIG. 3E is a graph providing the percentages of the GFP⁺ cells in the tamoxifen-labeled sham heart receiving 7 day treatment of vehicle or PGE2. FIG. 3F is a graph providing the ejection fraction percentages. Echocardiography was performed at one and two months post-infarction to assess the heart function. *, P<0.05; **, P<0.01; N.S., not significant. Sample size is indicated in the bar chart. Data are presented as the mean±s.e.m. These results demonstrate that inflammation response is required for cardiomyocyte replenishment after MI and PGE2, but not PGI2, treatment promotes this effect.

FIGS. 4A-4E evidence that PGE2 restores the ability of aged mice to replenish lost cardiomyocytes by modulating TGF-β pathway. Fourteen days post-MI, the hearts from aged mice (>18 months) were stained for GFP or β-Gal to evaluate the status of cardiomyocyte replenishment by endogenous stem cells at the border zone. FIG. 4A provides graphs of the percentages of GFP⁺ and β-Gal⁺ cardiomyocytes at the border zone that were quantified and statistically analyzed. FIG. 4B provides graphs of the percentages of GFP⁺ and β-Gal⁺ cardiomyocytes at the remote area that were quantified and statistically analyzed. FIG. 4C provides graphs of the fold change of TGF-β1 expression. Quantitative RT-PCR was performed to examine the effect of PGE2 on the expression of TGF-β1 in the infarcted region of MI heart in young and aged mice on day 3 and day 7 after injury. n≧3. FIG. 4D provides graphs of the percentages of GFP⁺ and β-Gal⁺ cardiomyocytes at the border zone that were quantified and statistically analyzed. Vehicle (DMSO) or TGF-β Type I Receptor inhibitor (ALK5i) was given to old MerCreMer/ZEG (M/Z) mice one day before and continuously for 10 days after MI surgery. The hearts were harvested on day 14 post-MI for GFP and β-Gal staining analysis. FIG. 4E provides graphs of the percentages of GFP⁺ and β-Gal⁺ cardiomyocytes at the remote area were quantified and statistically analyzed. *, P<0.05. **, P<0.01; N.S., not significant. Sample size is indicated in the bar chart. Data are presented as the mean±s.e.m. These results demonstrate that PGE2 treatment or inhibition of TGF-β signaling rescue the impairment of post-MI cardiomyocyte replenishment in aged mice.

FIGS. 5A-5B evidence that expression of stem cell marker gene Sca-1 changes in response to PGE2 treatment. Following surgery-induced heart injury, the infarct region of heart treated with indomethacin (Indo) or PGE2 was excised at different time points for RNA extraction and quantitative RT-PCR analysis. FIG. 5A are graphs providing the fold changes of expression of the pan-murine cardiac stem cell marker gene Sca-1 in the infarct zone of young and aged hearts using quantitative PCR analysis. FIG. 5B are graphs showing the fold changes of expression of the indicated cardiac stem cell marker genes at the infarct zone of young hearts using quantitative RT-PCR analysis. These results demonstrate that PGE2 treatment enhances the expression of Sca-1 (cardiac stem/progenitor cell marker) in the MI heart in both young and aged mice.

FIG. 6 provides graphs evidencing that PGE2 alters expression of cardiac transcription factor gene Nkx2.5 after myocardial infarction. Expression of the cardiac transcription factor genes in both infarcted and remote regions of injured hearts was analyzed by quantitative RT-PCR. The fold change is a relative quantification normalized to the sham control. ***, P<0.001. n≧3. Data are presented as the mean±s.e.m. These results demonstrate that PGE2 treatment also enhances the expression of Nkx2.5 (another cardiac stem/progenitor cell marker) in the MI heart in young mice.

FIGS. 7A-7C evidence that PGE2 improves cardiac differentiation ability of Sca-1⁺ cells after heart injury. FIG. 7A is a graph providing the percentages of Sca-1⁺/GFP⁺ cells on day 3 post-MI that were quantified by flow cytometry. Following myocardial infarction (MI) surgery, the MerCreMer/ZEG (M/Z) mice were injected with 80 μg/g tamoxifen (TAM) per day for 3 days with or without an additional PGE2 treatment. The sham control was also treated with the same dosage of tamoxifen and PGE2 simultaneously for 3 days. The cardiac small cells were isolated for flow cytometric analysis of Sca-1⁺/GFP⁺ cells on day 3 post-surgery. Mice that did not receive tamoxifen injections after the MI surgery served as negative control (no TAM). n≧4. FIG. 7B is a graph providing the fold change of the expression level of Nkx2.5 in the absence or presence of PGE2 following injury using quantitative RT-PCR. The cardiac Sca-1⁺ cells were isolated on day 3 after injury. FIG. 7C is a graph providing the percentages of Sca-1⁺/α-MHC⁺ cells after injury. Following the MI or sham surgery, the cardiomyocyte-depleted small cells from the wild-type (WT) or Sca-1-GFP transgenic mice were isolated on day 3 after surgery. The cardiac small cells were stained for α-MHC, a mature cardiomyocyte marker, to examine the percentage of Sca-1⁺/α-MHC⁺ cells after injury. These results demonstrate that PGE2 treatment increases cardiac differentiation ability of Sca-1⁺ cells in the MI heart.

FIGS. 8A-8D evidence that PGE2 may act through EP2-dependent signaling pathway to modulate cardiac differentiation of the Sca-1⁺ cell in vivo. FIG. 8A are graphs providing the fold changes in expression of the PGE2 receptors EP1, 2, 3 and 4 in response to different drug treatments at the infarct region of the MI heart as examined by quantitative RT-PCR. FIG. 8B are graphs providing the percentages of GFP⁺ and β-Gal⁺ cardiomyocytes at the border zone of the young heart with or without cell injection after MI was counted and statistically analyzed. Following MI surgery, the WT or EP2 deficient (EP2^{-/-}) cardiac Sca-1⁺ cells were injected intra-myocardially into the injured M/Z hearts. On day 14 post-infarction, the hearts were harvested for quantification of GFP⁺ or β-Gal⁺ cardiomyocytes. FIG. 8C are graphs providing the percentages of GFP⁺ and β-Gal⁺ cardiomyocytes at the remote region of the young heart with or without cell injection after MI that were quantified and statistically analyzed. FIG. 8D provides the percentage of small cells co-stained with mature sarcomeric structure following vehicle or PGE2 treatment. The cardiomyocyte-depleted small cells were allowed for attachment for 3 days, followed by PGE2 treatment (10 μm) for another 3 days. Sample size is indicated in the bar chart. *, P<0.05. **, P<0.01, ***, P<0.001; N.S., not significant. Data are presented as the mean±s.e.m. These results demonstrate that PGE2 activates Sca-1 cardiac stem/progenitor cells through the EP₂ receptor.

FIG. 9 provides graphs evidencing that PGE2 treatment changes the expression of inflammatory cytokines in the infarcted region of hearts from young mice. Following MI injury, the heart infarct areas were excised to analyze the expression of inflammatory cytokines, including MMP2, MMP9, TNF-α, IL-1β and IL-6. The fold change is a relative quantification normalized to the sham control. n≧3. Data are presented as the mean±s.e.m. These results demonstrate that PGE2 treatment alters the expression profiles of the major inflammatory cytokines in the MI hearts.

FIGS. 10A-10D evidence that PGE2 modulates post-infarction inflammatory response in the myocardium. FIG. 10A are graphs showing the fold changes in expression of T cell marker CD3, B cell marker B220 and myeloid cell marker CD11b in the infarct region of young mice on day 3 post-infarction, with or without PGE2 treatment using quantitative RT-PCR. n≧3. FIG. 10B are graphs showing the fold changes in expression of T cell marker CD3, B cell marker B220 and myeloid cell marker CD11b in the infarct region of aged mice on day 3 post-infarction, with or without PGE2 treatment using quantitative RT-PCR. n≧3. FIG. 10C are graphs providing the percentages of total CD11b⁺ cells, M1 (CD11b⁺/CD11c⁺) and M2 (CD11b⁺/CD206⁺) macrophages in young mice that were quantified and analyzed. Heart sections of young mice on day 3 post-infarction with or without PGE2 treatment were co-stained with CD11b/CD11c or CD11b/CD206, which represents the M1 and M2 type macrophages, respectively. Quantification is presented as a percentage calculated by dividing the number of CD11b⁺ cells or double-positive cells by the total number of cells. n≧3. FIG. 10D are graphs providing the fold changes in expression of IL-10 in response to PGE2 treatment on day 3 after infarction at the injured region of young and old mice using quantitative RT-PCR analysis. n≧3. *, P<0.05, **, P<0.01. Data are presented as the mean±s.e.m. These results demonstrate that PGE2 treatment decreases the amount of pro-inflammatory M1 macrophage and increases the amount of anti-inflammatory M2 macrophage in the MI heart. PGE2 treatment also promotes the expression of anti-inflammatory cytokine IL-10.

FIG. 11 shows the primer sequences used for quantitative or semi-quantitative PCR. From left to right, top to bottom, the sequences are SEQ ID NOs: 1-48.

### DETAILED DESCRIPTION OF THE INVENTION

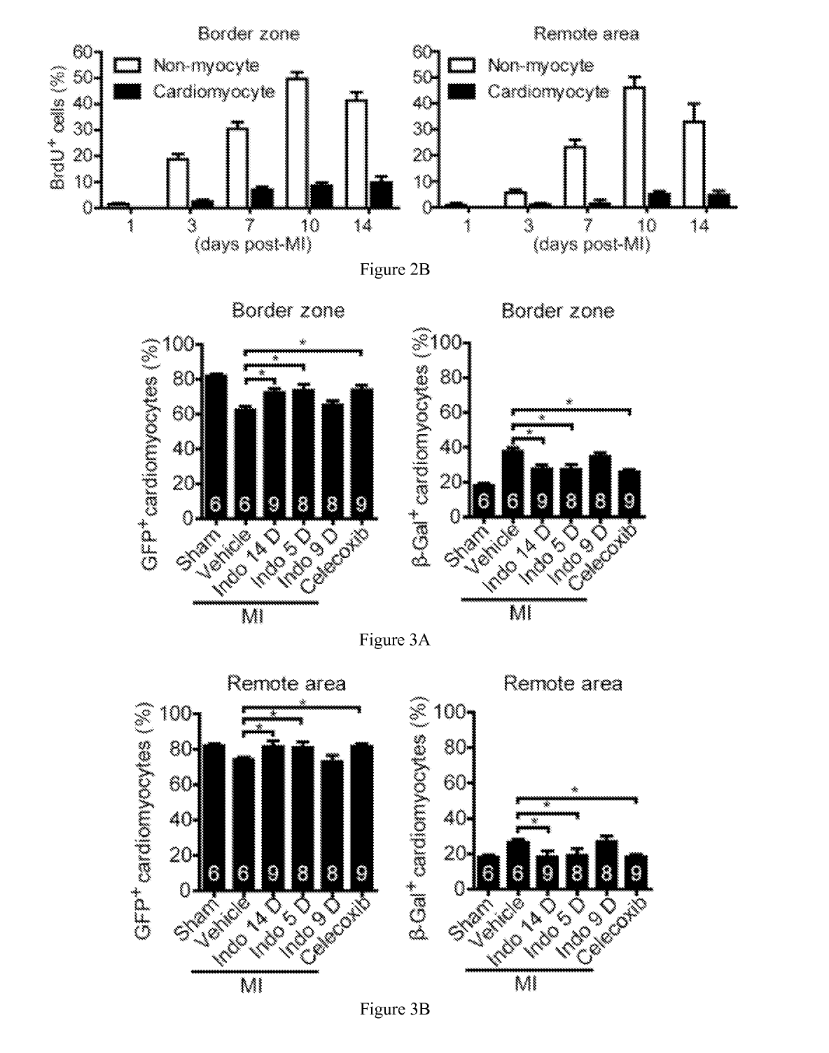

Contrary to the prior art teachings, the experiments herein evidence that lost and/or damaged cardiomyocytes in injured cardiac tissues may be replenished with newly generated cardiomyocytes from endogenous stem and progenitor cells and this cell-mediated cardiomyocyte replenishment process saturates within about 10 days after the injury. In addition, the experiments herein show that the number of pre-existing cardiomyocytes that undergo proliferation is relatively low at early time points, e.g., within about 2 weeks after injury. Although pre-existing cardiomyocytes may play a role in cell replenishment (only a small portion of new cardiomyocyte formation is from pre-existing cardiomyocyte proliferation at the early stage of post-MI, FIG. 2), the experiments herein evidence that endogenous stem and progenitor cells can result in a significant replenishment of lost and/or damaged cardiomyocytes in cardiac tissues during the early stages after injury thereto. As used herein, the terms “replenish” and its related forms are used interchangeably with the terms “repopulate” and its related forms to mean, in the context of the present invention, that some or all of the cardiomyocytes that are lost or damaged in a cardiac tissue are replaced with newly generated cardiomyocytes.

Therefore, the present invention provides methods and compositions for treating damaged and/or diseased cardiac tissue in subjects such as animals and humans, preferably human subjects. In particular, the present invention provides methods and compositions for replenishing lost and/or damaged cardiomyocytes in cardiac tissue. As the experiments evidencing the replenishment of lost and/or damaged cardiomyocytes occurs within about 10 days after the injury were conducted in mouse models and the cell-mediated cardiomyocyte replenishment process is relatively longer in humans, it is expected that the methods and compositions according to the present invention may replenish lost and/or damaged cardiomyocytes in human subjects within about 7-10 days, within about 30 days, and at most, within about 3 months, of an event which will likely result in injury and/or damage to the cardiac tissue of the subject, after the event causing the lost and/or damaged cardiomyocytes. As used herein, “cardiac tissue” refers to one or more tissues (e.g., endocardium, myocardium, and epicardium) and cells (e.g., cardiomyocytes, cardiac fibroblasts, endothelial cells, and vascular smooth muscle cells) of a heart, preferably a mammalian heart, more preferably a human heart. In some embodiments, the cardiac tissue is injured or damaged by a myocardial infarction (MI). In some embodiments, the cardiac tissue is injured or damaged by ischemia, hypoxia, coronary artery disease (CAD), cardiomyopathy, myocardial infarction, atherosclerosis, heart failure, congenital heart diseases, valvular heart diseases, ischemic heart diseases, and other conditions which damage the heart tissue such as viral infection or trauma.

As disclosed herein, cardiomyocyte replenishment by endogenous stem and progenitor cells may be induced, increased, and/or enhanced by attenuating TGF-β1 signaling and/or modulating EP2 receptor activity. Specifically, blocking the inflammatory reaction with COX-2 inhibitors reduced the capability of endogenous stem and progenitor cells to repopulate the lost or damaged cardiomyocytes. Surprisingly, however, treatment with PGE2 was found to induce, increase, and/or enhance cardiomyocyte replenishment by endogenous stem and progenitor cell in young animal models as well as recover cell renewal by attenuating TGF-β1 signaling in aged animal models. Thus, in some embodiments, the present invention is directed to methods and compositions for replenishing lost and/or damaged cardiomyocytes in a subject which comprises inducing, increasing, and/or enhancing the activation and/or differentiation of endogenous stem and/or progenitor cell in the subject by attenuating or inhibiting the Transforming Growth Factor beta 1 (TGF-β1) signaling pathway and/or activating the prostaglandin E2 receptors (EP2) in the subject.

A compound that attenuates or inhibits the TGF-β1 signaling pathway is referred to herein as a “TGF-β1 signaling inhibitor”. TGF-β1 signaling inhibitors include SB 431542, a potent and selective inhibitor of ALK5; LY2157299, a potent TGFβ receptor I inhibitor; LDN193189, a selective inhibitor of ALK2 and ALK3; SB 525334, a potent and selective inhibitor of TGF-β1 (ALK5); LY2109761, a selective TGF-β receptor type I/II dual inhibitor; SB505124, a selective inhibitor of ALK4 and ALK5; GW788388, a potent and selective inhibitor of ALK5; Pirfenidone, an inhibitor of TGF-β bioactivity by affecting TGF-β2 mRNA expression and processing of pro-TGF-β in CCL-64 cells; Y364947, a potent ATP-competitive inhibitor of TGFβR-I; IDT-1, a specific TGF-β inhibitor; E-616452 and E-616451, TGF-β1 kinase inhibitors; and the like, including those as described in U.S. Pat. Nos. 5,958,411; 6,329,500; 6,500,920; 6,673,341; 7,173,002; 7,420,050; 7,407,958; and 8,110,655. Examples of compounds that activate EP2 receptors include prostaglandin E2 (PGE2, (5Z,11α,13E,15S)-7-[3-hydroxy-2-(3-hydroxyoct-1-enyl)-5-oxo-cyclopentyl]hept-5-enoic acid) and analogues thereof, and prostaglandin E2 agonists (PGE2 agonists) known in the art such as butaprost (CAS 69685-22-9), CP-533,536 (Li et al. (2003) J Bone Miner Res. 18:2033-2042), and ONO-AE1-259-01 (Mori, et al. (2009) Eur. J. Pharmacol, 616:64-67), and those disclosed in U.S. Pat. Nos. 5,663,417; 6,046,236; 6,426,359; 6,531,485; 7,053,085; 7,238,710; 7,109,223; 7,238,710; and the like.

As disclosed herein, PGE2 increases, as well as rescues, cardiomyocyte replenishment by endogenous stem and progenitor cells after injury to cardiac tissues. Therefore, in some embodiments, the methods and compositions of the present invention employ using at least one PGE2 compound. In particular, in some embodiments, the compositions comprise at least one PGE2 compound. Similarly, in some embodiments, the methods of the present invention comprise administering at least one PGE2 compound to the subject being treated. As used herein, a “PGE2 compound” includes PGE2 its analogues and salts and derivatives thereof, derivatives of the receptors of prostaglandin E2 (EP1-4) (e.g., sulprostone ((Z)-7-[(1R,3R)-3-hydroxy-2-[(E,3R)-3-hydroxy-4-phenoxybut-1-enyl]-5-oxocyclopentyl]-N-methylsulfonylhept-5-enamide), butaprost (CAS 69685-22-9), enprostil (ethyl 7-[(1S,2S,3S)-3-hydroxy-2-[(S,E)-3-hydroxy-4-phenoxybut-1-enyl]-5-oxocyclopentyl]hepta-4,5-dienoate) and ONO-4819 (Marui et al. (2006) J. Thoracic and Cardiovas. Surgery 131:587-593), and compounds having as part of their structural backbone the following structural formula (I), which may or may not be substituted:

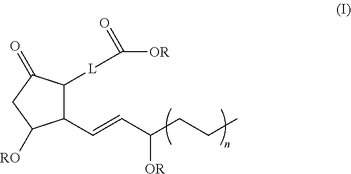

wherein n is an integer between 1 and 10, preferably between 1 and 5, more preferably 2; L is a linker which can be a substituted or unsubstituted alkyl, a substituted or unsubstituted alkenyl; and the R's are each independently H, a halogen, a substituted or unsubstituted C₁₋₆ alkyl, or a substituted or unsubstituted C₁₋₆ alkenyl; and acid, bases, and salts thereof. This means that the PGE2 compound having structural formula I as its core structure may contain any number of substituents so long as it exhibits some ability to bind and/or activate a prostaglandin E2 receptor, preferably a human prostaglandin E2 receptor (which may be a recombinant receptor), as compared to a control. Other PGE2 compounds according to the present invention include PGE2 analogs as disclosed in U.S. Pat. Nos. 5,663,417; 6,046,236; 6,426,359; 6,531,485; 7,053,085; 7,238,710; 7,326,732; 7,109,223; 7,238,710; 8,063,240; and the like.

In some embodiments, the present invention provides methods of treating a subject having damaged cardiac tissue, trauma to one or more cardiac tissues, and/or cardiovascular diseases and methods of inhibiting and/or reducing the amount of damaged or scarred cardiac tissue in subjects which comprises administering the at least one PGE2 compound to the subject. As used herein, “a cardiovascular disease” refers to heart diseases such as coronary artery disease (CAD), cardiomyopathy, myocardial infarction, ischemia, atherosclerosis, heart failure, congenital heart diseases, valvular heart diseases, ischemic heart diseases, and other conditions which damage the heart tissue such as viral infection or trauma, and vascular diseases such as peripheral artery occlusive diseases, Raynaud's phenomenon, Buerger's disease, and other connective tissue disorder associated vascular inflammation or damage. The at least one PGE2 compound may be administered to the cardiac tissue before, during, and/or after the onset of the cardiovascular disease or trauma, e.g., surgery, which will likely result in damage to the cardiac tissue if left untreated. In some embodiments, the at least one PGE2 compound is administered orally. In some embodiments, the at least one PGE2 compound is systemically administered, e.g., intravenously, or into the cardiac tissue to be treated. Thus, the injection routines include (1) epicardial injection by surgical, echo-guided or endoscope-assisted approach, (2) transendocardial injection by a catheter or during open heart surgery or (3) intravascular injection. In some embodiments, the PGE2 is applied directly on the cardiac tissue to be treated, e.g., during open heart surgery. In some embodiments, the at least one PGE2 compound is administered orally to the subject before, during, and/or after an event that will likely result in injury to the cardiac tissue.

In some embodiments, the at least one PGE2 compound is administered in an effective amount. As used herein, an “effective amount” is the amount of the at least one PGE2 compound which results in the desired effect (e.g., binding and/or activation of the PGE2 receptors) as compared to a control such as a placebo. An effective amount may be readily determined by standard methods known in the art. The dosages to be administered can be determined by one of ordinary skill in the art depending on the clinical severity of the condition to be treated and the age and weight of the subject. Effective amounts of PGE2 range from about 0.001 to about 1.0, preferably to about 0.5, more preferably to about 0.1 mg/kg body weight. Thus, in some embodiments, preferred effective amounts of a PGE2 compound ranges from about 0.001 to about 1.0, preferably to about 0.5, more preferably to about 0.1 per kg body weight. One skilled in the art may readily determine the effective amounts for human subjects using the methods described herein and/or drawing correlations from these animal models.

Treatment of a subject with the at least one PGE2 compound according to the present invention can include a single treatment or a series of treatments. It will be appreciated that the actual dosages will vary according to the particular composition, the particular formulation, the mode of administration, and the particular subject and condition being treated. It will also be appreciated that the effective dosage used for treatment may increase or decrease over the course of a particular treatment. Optimal dosages for a given set of conditions may be ascertained by those skilled in the art using conventional dosage-determination tests in view of the experiments herein. Changes in dosage may result and become apparent by standard diagnostic assays known in the art.

The pharmaceutical compositions of the invention may be prepared in a unit-dosage form appropriate for the desired mode of administration. As an example, compositions comprising the at least one PGE2 compound may be administered to a subject either by injection or orally before, during, and/or after an event which is likely to cause injured and/or damaged cardiac tissue. It will be appreciated that the preferred route will vary with the condition and age of the subject, the nature of the condition to be treated, and the given composition.

In addition to the at least one PGE2 compound, the compositions of the present invention may further comprise an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration and known in the art. Except insofar as any conventional media or agent is incompatible with the active compound, use thereof in the compositions is contemplated.

As set forth in the experiments herein, the period of cardiomyocyte replenishment by endogenous stem and progenitor cells is up to about 7-10 days after injury to the cardiac tissue. Therefore, in some embodiments, one or more compositions according to the present invention (e.g., compositions comprising at least one PGE2 compounds, compositions that attenuate or inhibit the TGF-β1 signaling pathway, compositions that activate EP2 receptors) are administered to the subject within about 3 months, preferably within about 30 days, more preferably within about 7-10 days, of an event which will likely result in injury and/or damage to the cardiac tissue of the subject.

In addition, experiments herein suggest that cardiac Sca-1⁺ cells are the major cell population that is responsive to PGE2 and that PGE2 may regulate endogenous stem cell activity directly through the EP2 receptor or indirectly by modulating its micro-environment in vivo.

The following examples are intended to illustrate but not to limit the invention.

### Materials and Methods

### Mouse Breeding

All experiments involving animals were conducted in accordance with the Guide for the Use and Care of Laboratory Animals, and all animal protocols have been approved by National Cheng Kung University. Sca-1⁻ GFP, B6.Cg-Tg(Ly6a-GFP)G5Dzk/J and EP2^{-/-}, B6.129-Ptger2^{tm/Brey}/J mice were obtained from Jackson Laboratory. The double transgenic MerCreMer/ZEG (M/Z) mice were generated by crossbreeding MerCreMer and Z/EG mice (Jackson Laboratory), which have C57BL/6SV129 and C57BL/6J (N7) background strains, respectively. The MerCreMer mice contain a tamoxifen-inducible Cre recombinase fusion protein driven by the cardiomyocyte-specific α-MHC promoter. In Z/EG mice, GFP replace constitutive β-Gal expression after the removal of a LoxP-flanked stop sequence by Cre.

### Surgery and Echocardiography

M/Z mice were subjected to experimental myocardial infarction (MI) one month after the last tamoxifen injection. MI was generated by ligating the left anterior descending coronary artery at 2-3 mm distal to the left atrial appendage. For cell transplantation experiments, intramyocardial injections were performed immediately after coronary ligation. A total of 6×10⁵ cardiac Sca-1⁺ cells were injected per mouse in two divided injections (5 μl/injection). For BrdU labeling, the mice were subcutaneously implanted with osmotic minipump (Alzet) at the time of surgery and 40 μg/μl of BrdU (Sigma) was administrated. For immunohistological studies, mice were sacrificed and the hearts were harvested at different time points following experimental MI surgery. Cardiac performances were assessed by echocardiography at 28 and 56 days after operation using Vevo 770 (Visualsonics, Toronto, Canada).

### Drug Treatment

To induce Cre recombination to achieve GFP labeling of cardiomyocytes, tamoxifen (Sigma) was dissolved in sunflower oil (Sigma) at a concentration of 5 mg/ml. The tamoxifen solution was injected intraperitoneally into M/Z mice daily at a dosage of 40 μg per 1 g body weight for 14 days, and the same dosage was injected twice daily for short-term labeling using methods known in the art (Laugwitz, K.-L. et al. Nature 433, 647-653 (2005)). All experimental conditions were optimized prior to the PGE2, indomethacin and TGF-β Type I Receptor Kinase Inhibitor II (ALK5 Inhibitor II, ALK5i, Merck) treatments. The mice treated with PGE2 or PGI2 (Vegiopoulos, A. et al. Science 328, 1158-1161 (2010)) (both from Sigma) were injected intraperitoneally with 3.33 ng of drug per 1 g of body weight dissolved in absolute ethanol twice daily. For the indomethacin treatment, mice were fed with water containing indomethacin (Sigma, 15 μg/ml) for different periods of time. The indomethacin-containing water was changed every 3 days. The mice subjected to the celecoxib (Lyons, T. R. et al. Nat. Med. 17, 1109-1115 (2011)) (Sigma) treatment were injected intraperitoneally with 5 μg of drug per 1 g of body weight daily. For ALK5i treatment, aged mice were injected intraperitoneally once per day with 1 μg of drug per 1 g of body weight one day before surgery and continuously until day 10 post-MI. Celecoxib and ALK5i were dissolved in ethanol and DMSO, respectively.

### GFP⁺ or β-Gal⁺ Cardiomyocyte Counting

All of the cellular quantifications were performed double-blindly to minimize personal bias. To achieve this, photo taken from the scar tissue was avoided so that the personnel performing cell quantification did not know if the photos were taken from the border zone or the remote area. For the cardiomyocyte cell counts, 3 sections from each heart, and 2 infarction border zones and 1 remote area from each section were analyzed at a magnification of 200× by light microscopy. Cells with visible sarcomere structures were analyzed, and the average number of cells counted was 171.8±5.8 per photo image. For the small cardiac cell counts, more than five sections from each heart were analyzed at a magnification of 400× using fluorescence microscopy. The average number of cells counted was 17.01±0.99 per photo image, and more than one hundred and fifty cells were analyzed from each heart. As quantification result is the averaged values calculated from the pictures taken from six border zone sections per heart, personal variation has been minimized.

### Software-Based Image Analysis

The immunohistochemically stained images were subjected to color separation to produce two gray images in which the areas occupied by DAB⁺ and DAB⁻ cells were separated. The empty area containing neither DAB⁺ nor DAB⁻ cells was subtracted from the total area following the separation of the DAB⁺ and DAB⁻ areas (μm²) so that the empty area did not interfere with the DAB⁻ cell counts.

### Immunohistochemistry and Immunofluorescence Microscopy

The harvested hearts were fixed with 4% paraformaldehyde and embedded in paraffin. The sections were then immunostained with the following primary antibodies: mouse anti-GFP (1:500, MBL), rabbit anti-GFP (1:200, Abcam or GeneTex), Donkey anti-GFP (1:200, Abcam), rabbit anti-β-Gal (1:500, Invitrogen), rabbit anti-Nkx2.5 (1:50, Abcam), mouse anti-BrdU (1:100, Roche), mouse anti-cTnT (1:100, DSHB), rabbit anti-cTnI (1:100, Abcam) and rat anti-Sca-1-PE (1:500, BD Bioscience). A DAB substrate kit (Vector Laboratories) was used for immunohistochemistry and appropriate secondary antibodies (Invitrogen or Abcam) were used for visualization under a fluorescence microscope. The plasma membrane was immunostained with wheat germ agglutinin (WGA, 5 μg/ml, Invitrogen) and 4,6-diamidino-2-phenylindole (DAPI, 1 μg/ml; Sigma) was used for nucleus staining. For preparation of frozen section, the hearts were dehydrated for 6 hours in 15% sucrose solution and followed by overnight in 30% sucrose solution. After dehydration, the organs were embedded in tissue freezing medium at −20° C. The frozen sections were immunostained with the following primary antibodies: rat anti-CD11b (1:700, BioLegend), mouse anti-CD68 (1:10,000, Abcam), rat anti-mouse CD206 (1:500, AbD Serotec) and hamster anti-mouse CD11c (1:1,000, Biolegend). The appropriate secondary antibodies (Invitrogen or Jackson ImmunoResearch) were used for visualization under a fluorescence microscope. Respective isotype controls (BD Biosciences or GeneTex) were used as negative controls.

### Extraction and Preparation of Total RNA for Semi-Quantitative and Quantitative PCR

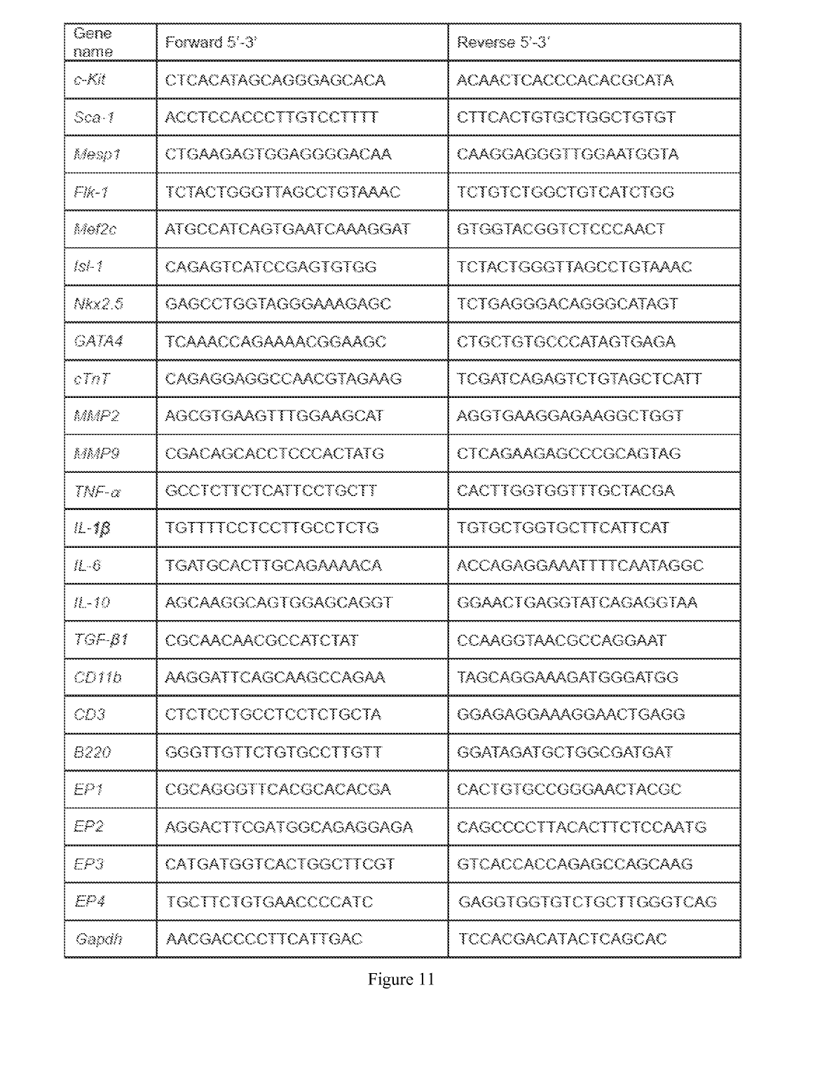

The total RNA isolated from the ischemic region or remote area of MI hearts was reverse transcribed using the SuperScript III (Invitrogen) system according to the manufacturer's protocol. For quantitative PCR, the SYBR Green reagent (Maestrogen) was used according to the manufacturer's protocol. The analysis of relative gene expression was performed using the 2̂[−delta delta Ct] method and sequence-specific primers designed for semi-quantitative PCR and real-time RT-PCR (FIG. 11).

### Flow Cytometry, Cell Isolation, Culturing and Immunocytochemistry Staining

Cardiomyocyte-depleted cardiac small cells were prepared as previously described with some modifications (Oh, H. et al. Proc. Natl. Acad. Sci. USA 100, 12313-12318 (2003); and Pfister, O. et al. Circ. Res. 97, 52-61 (2005)). Specifically, instead of using a 70 μm and then a 45 μm strainer to isolate the cardiomyocyte-depleted cardiac small cells, the cells were filtered through a 45 μm strainer directly after enzyme digestion. The minced heart tissue was digested with 0.1% collagenase B (Roche Molecular Biochemicals), 2.4 U/ml dispase II (Roche Molecular Biochemicals) and 2.5 mmol/L CaCl₂ at 37° C. for 30 minutes and then filtered through a 45-μm filter. For isolation of cardiac Sca-1⁺ cells, the cardiac small cells were incubated with the Phycoerythrin (PE)-conjugated Sca-1⁺ antibodies (BD Bioscience) at 4° C. for 30 minutes. The PE-labeled Sca-1⁺ cells were then sorted by the magnetic particles against PE (BD Biosciences). Incubation of cardiomyocyte-depleted small cells with anti-Sca-1-PE antibody and magnetic cell sorting were omitted for isolation of Sca-1⁺ cells from the Sca-1-GFP transgenic mice. After isolation, the cells were fixed in 2% paraformaldehyde for 20 minutes and followed by 1 hour blocking in PBS with 1% BSA. 5×10⁶ of cell were stained in 0.25 μg of anti-α-MHC antibody (Abcam) in 100 μl of blocking buffer for 30 minutes at room temperature in dark, and followed by staining with anti-mouse-PE (1:200, Invitrogen). Respective isotype controls (BD Biosciences or GeneTex) were used as negative controls. Flow cytometry was performed using the FACSCanto™ (BD). The FACSDiva™ (BD) and FlowJo software was used for data analysis. For cell culture, 3×10⁵ cells were plated per well in a 6-well dish coated with 200 μg/ml fibronectin (Millipore). The cells were cultured in Iscove's Modified Dulbecco's Medium (IMDM) (Invitrogen) supplemented with 10% FBS and penicillin/streptomycin at 37° C. (Matsuura, K. et al. J. Clin. Invest. 119, 2204-2217 (2009)). The culture medium was changed 3 days after plating and the cells were treated with PGE2 (10 μm) for another three days. On day 10, immunocytochemistry (ICC) staining was performed. For ICC staining, the cells were fixed in 2% paraformaldehyde and blocked in 1% BSA. The cells were stained with the cTnT (1:100, DSHB) overnight at 4° C. and membrane dye WGA (5 μg/ml, Invitrogen) at room temperature for 10 minutes.

### Data Analysis

The results were statistically analyzed using either one-way ANOVA or t-tests. A result was considered to be statistically significant if the P value was <0.05.

### Time Period of Cardiomyocyte Replenishment

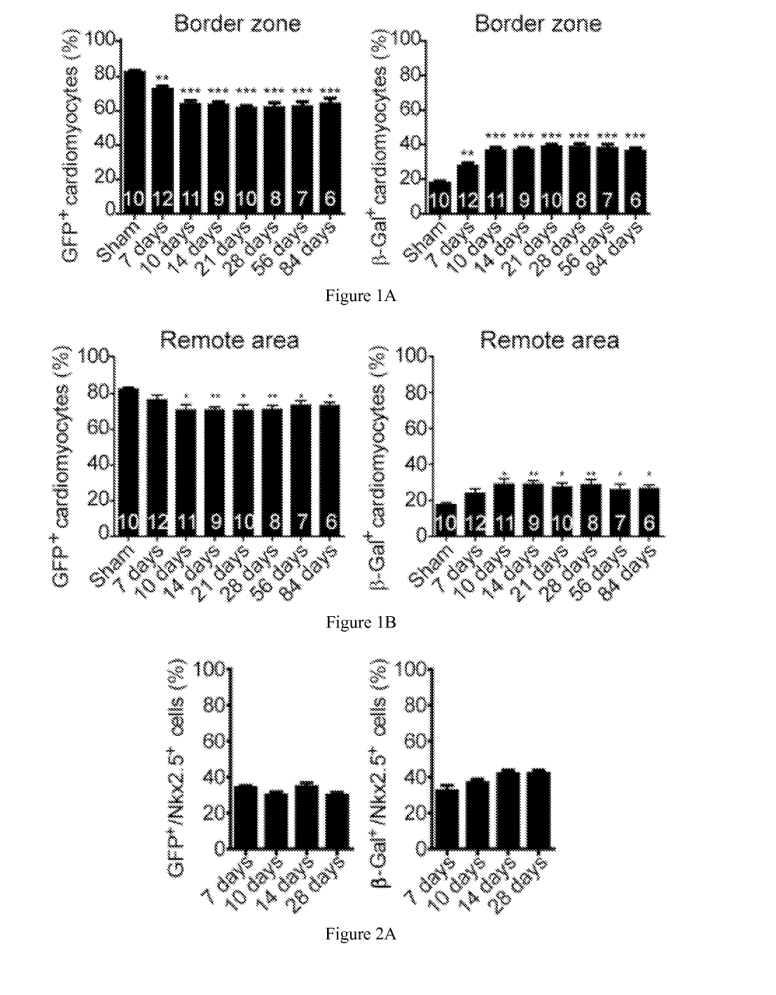

To determine the most critical time period for cardiomyocyte replenishment, the cardiac specific tamoxifen-inducible Cre-LoxP transgenic MerCreMer/ZEG (M/Z) mice with the use to trace endogenous stem/progenitor cell-driven cardiomyocyte replenishment upon injury in vivo. Analysis of GFP⁻ cardiomyocytes at the border zone revealed that about 10% of the stem cell-derived GFP⁻ cardiomyocytes were renewed within 7 days post-MI compared to the sham group (P<0.01; FIG. 1A). Furthermore, the degree of cell replacement saturated at about 18.9% on day 10 post-MI (P<0.001) and leveled off until the third month (FIG. 1A). This result was consistent with the β-Gal staining data (FIG. 1A). Examination of the regenerative efficiency at the remote area indicated that cardiomyocyte replenishment also saturated on day 10 after MI (FIG. 1B). Moreover, immunofluorescent staining was performed to quantify the number of cardiomyocytes labeled by BrdU and the β-Gal⁺ or GFP⁺ cardiomyocytes expressing Nkx2.5 (Sturzu & Wu, Circ. Res. 108, 353-364 (2011)) (FIG. 2). The results revealed that the number of BrdU⁺ cardiomyocytes increased on day 7 and reached a plateau on day 10 post-infarction. The same phenomenon was observed in the β-Gal⁺/Nkx2.5⁺ cardiomyocytes. These results are consistent with previous reports showing that transplanted or resident cardiac stem cells are capable of differentiating into cardiomyocytes within about 7-14 days after MI.

### Effect of PGE2 on Cardiomyocyte Replenishment

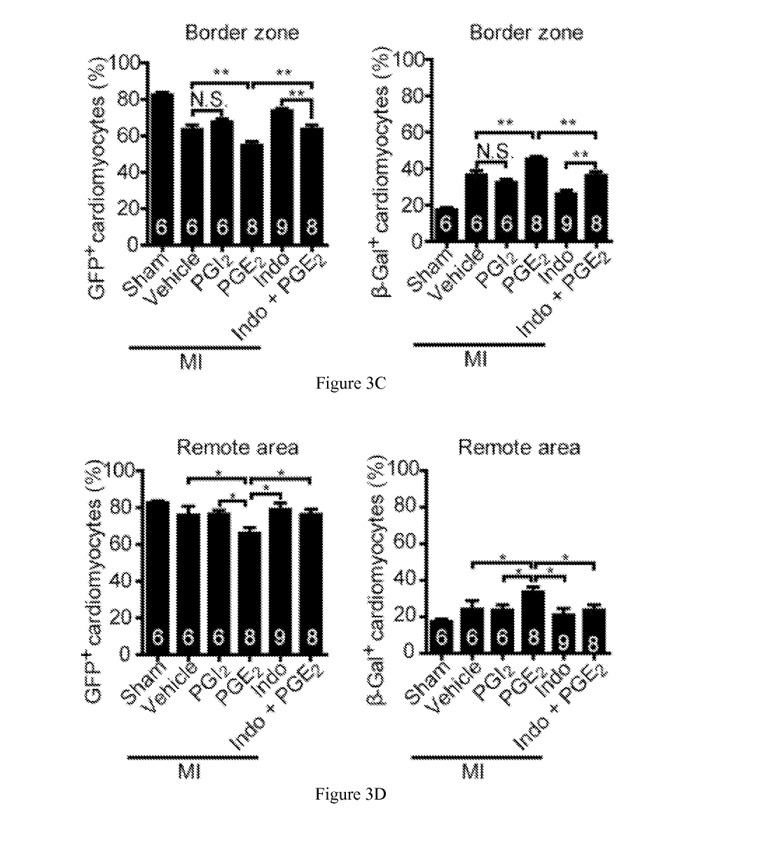

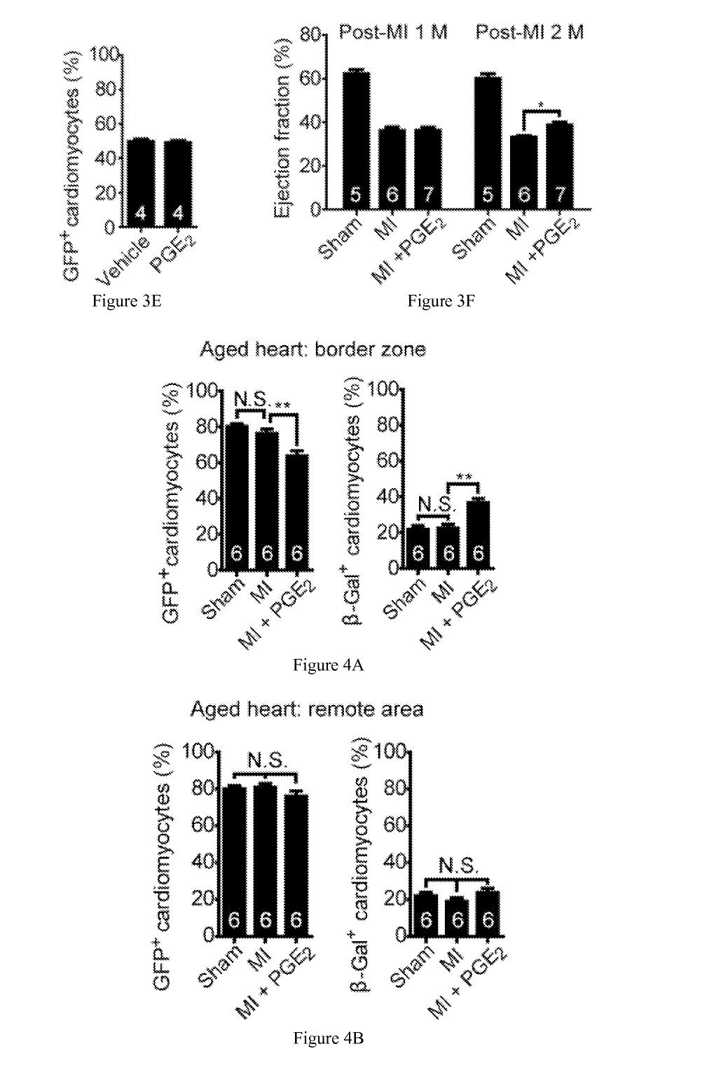

To explore the effect of MI-induced COX-2 expression and PGE2 production on cardiomyocyte repopulation, mice were treated with indomethacin, a non-selective COX pathway inhibitor. At the border zone, the cardiomyocyte restoration rate dropped by about 10% upon indomethacin administration (Indo 14D, P<0.05; FIG. 3A). Indomethacin also abrogated MI-induced cardiomyocyte replenishment in the remote area, where the inflammatory response was weaker than at the border zone (FIG. 3B). Further examination revealed that indomethacin given during the first 5 days post-MI (Indo 5 D) was sufficient to impair cardiomyocyte replenishment at the border zone (P<0.05). However, indomethacin had no significant effect on stem cell-dependent cardiomyocyte reconstitution when administered more than 5 days post-MI (Indo 9 D, FIGS. 3A and 3B). Indomethacin is a pan-COX inhibitor. Thus, indomethacin affects both COX-1 and COX-2 pathways. Following a 14-day treatment of Celecoxib, a selective COX-2 inhibitor, cardiomyocyte replenishment was effectively blocked (FIGS. 3A and 3B), thereby suggesting that the COX-2-dependent inflammatory response is critical for cardiomyocyte repopulation upon activation of stem cells and the duration of this signal is short.

Cardiomyocytes Replenishment after Injury

Whether COX-2 downstream effectors could promote cardiomyocyte replenishment from endogenous stem cells was examined. Mice were treated with prostaglandin I₂ (PGI₂) (Vegiopoulos, A. et al. Science 328, 1158-1161 (2010)) or prostaglandin E₂ (PGE2) (Goessling, W. Pt al. Cell 136, 1136-1147 (2009)). It was found that PGE2, but not PGI2, treatment significantly increased cardiomyocyte replenishment at the border zone by about 9% compared to the vehicle control (P<0.01; FIG. 3C) and PGE2 did not alter the GFP labeling efficiency before surgery (FIG. 3E). In addition, PGE2 was able to rescuecardiomyocyte replenishment by about 10% compared to indomethacin treatment alone (P<0.01; FIG. 3C). In other words, PGE2 treatment resulted in significant increase in the stem cell-derived cardiomyocytes, i.e., the β-Gal⁺ cardiomyocytes, in the injured heart treated with indomethacin-PGE2 treatment restored indomethacin-attenuated cardiomyocyte replenishment from endogenous stem cells. A similar trend was observed in the remote area (FIG. 3D). Furthermore, the cardiac function post-MI was improved by PGE2 treatment (FIG. 3F).

### Cardiomyocytes Replenishment in Aged Hearts

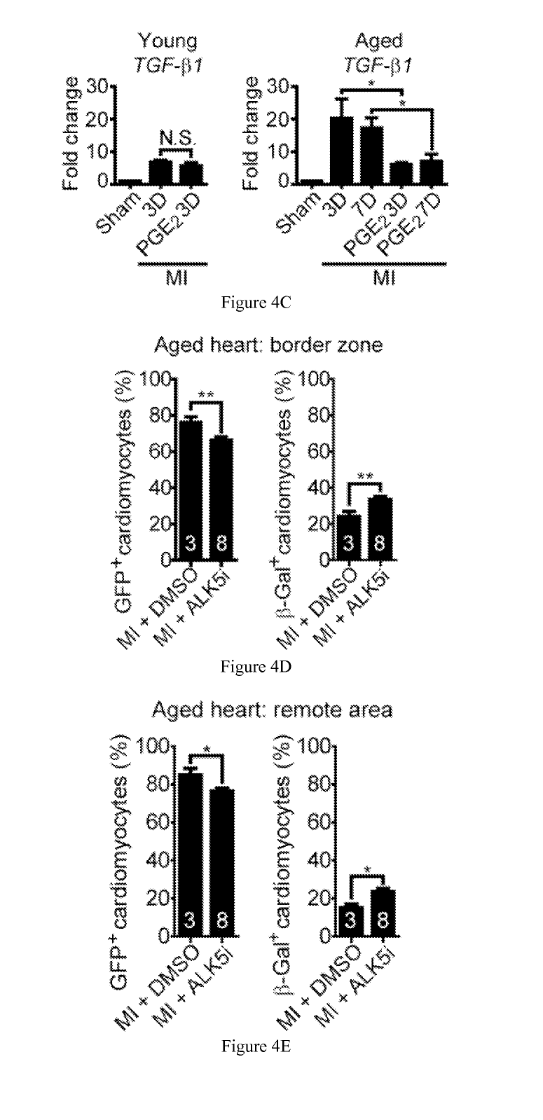

Because the aged heart loses its regenerative ability, the degree of cardiomyocyte regeneration was examined in old mice. In aged mice (>18 months), regardless of the same GFP labeling efficiency, MI itself did not induce evident cardiomyocyte replenishment at the border zone (FIG. 4A). Surprisingly, PGE2 treatment could successfully recover the attenuated stem cell-mediated cardiomyocyte reconstitution at the border zone (P<0.01 versus sham or MI) of aged hearts, but not in the remote area (FIGS. 4A and 4B). Expression of the aging-associated marker gene, transforming growth factor β-1 (TGF-β1) (Luo, S., et al. Cell 143, 299-312 (2010); and Carlson, M. E., et al. Nature 454, 528-532 (2008)) was observed to decline in PGE2-treated aged mice. However, its level in young mice was not significantly altered by PGE2 (FIG. 4C).

To examine the effect of TGF-β1 pathway on cardiomyocyte replenishment in aged mice, the animals were treated with the TGF-β Type I Receptor Kinase Inhibitor II (ALK5 Inhibitor II, ALK5i) (Ichida, J. K. et al. Cell Stem Cell 5, 491-503 (2009)). ALK5i was administered from one day before surgery and continued until day 10 post-MI. In comparison with the vehicle control group, ALK5i restored cardiomyocyte replenishment on day 14 post-MI (P<0.01, FIGS. 4D and 4E), and the percentage of GFP⁺ or β-Gal⁺ cardiomyocytes was similar to that observed in the young mice subjected to MI surgery only. This implies that high TGF-β1 activity may negatively regulate cardiomyocyte replenishment after MI in aged hearts. Therefore, PGE2 not only augments cardiomyocyte replenishment in young mice but also rescues the self-regenerative function in aged heart, thereby suggesting that the COX-2/PGE2 pathway is necessary to induce stem cell-driven cardiomyocyte replenishment.

### Sca-1⁺ Cells are the Major Population Responsive to PGE2 Treatment in Mice

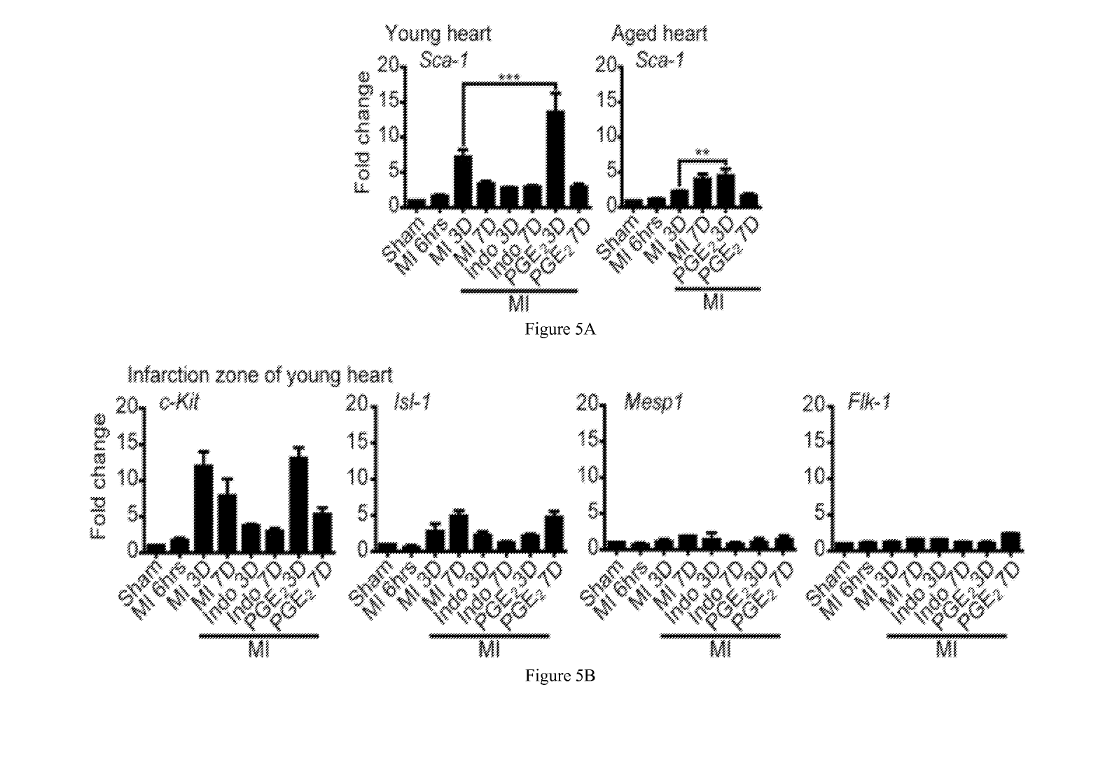

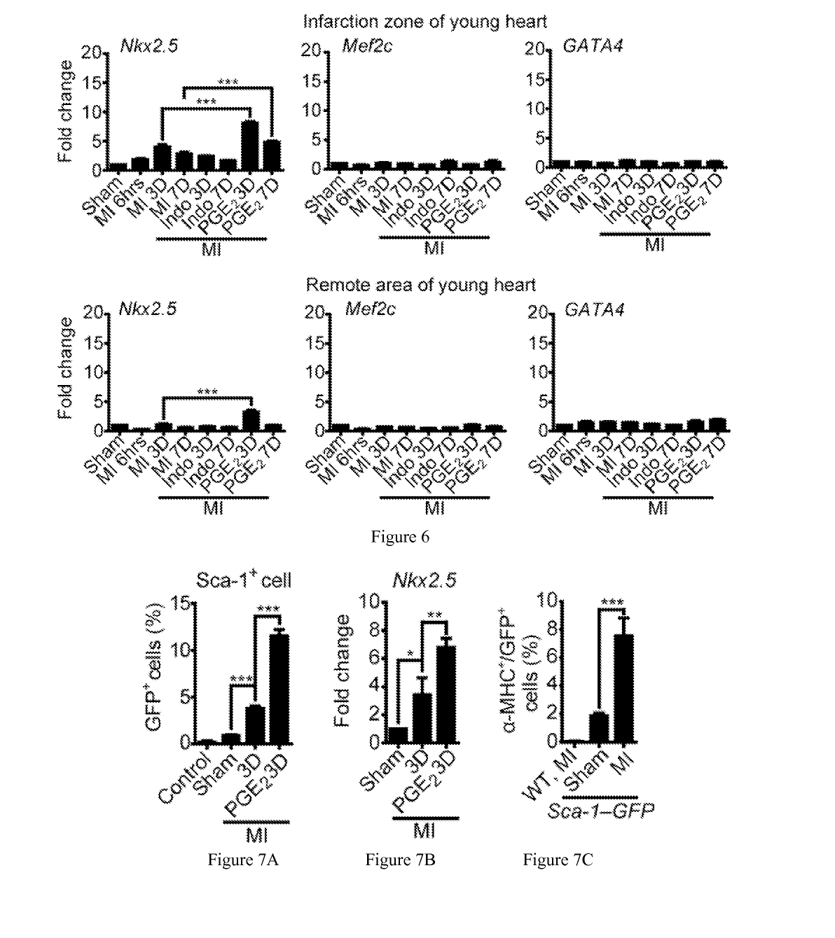

As Sca-1 is commonly expressed in various cardiac stem/progenitor cell populations in mice, the effect of PGE2 on stem cell-mediated cardiomyocyte replenishment by examining Sca-1⁺ cell activities was examined. Quantitative RT-PCR revealed that only Sca-1 expression peaked on day 3 post-MI and this level was further increased at the same time point upon PGE2 treatment but was repressed by indomethacin (FIGS. 5A and 5B). Expression of the cardiac transcription factors suggested that Nkx2.5 had similar expression pattern to that of Sca-1 at the infarct zone and the remote area (FIG. 6). In aged animals, Sca-1 expression was significantly lower at the same time point, suggesting that aging attenuates stem cell-dependent tissue repair. However, PGE2 restored Sca-1 expression to levels similar to those in young mice (FIG. 5A).

Because tamoxifen injection induces conversion of α-MHC⁺/β-Gal⁺ cells into α-MHC⁺/GFP⁺ cells in M/Z mice, whether PGE2 augments cardiac differentiation potential of Sca-1⁺ cells was examined by quantifying the percentage of Sca-1⁺/GFP⁺ cells. In addition to expanding the number of Sca-1⁺/GFP⁺ cells (P<0.001; FIG. 7A), PGE2 also elevated the expression of Nkx2.5 in Sca-1⁺ cells (FIG. 7B). Using Sca-1-GFP or Ly-6A-GFP transgenic mice (Boisset, J.-C. et al. Nature 464, 116-120 (2010)), the same result was obtained as that in the M/Z mice. Further, there was a significant increase in the α-MHC⁺/GFP⁺ cell population in the MI heart of Sca-1-GFP mouse as compared to the sham group (FIG. 7C).

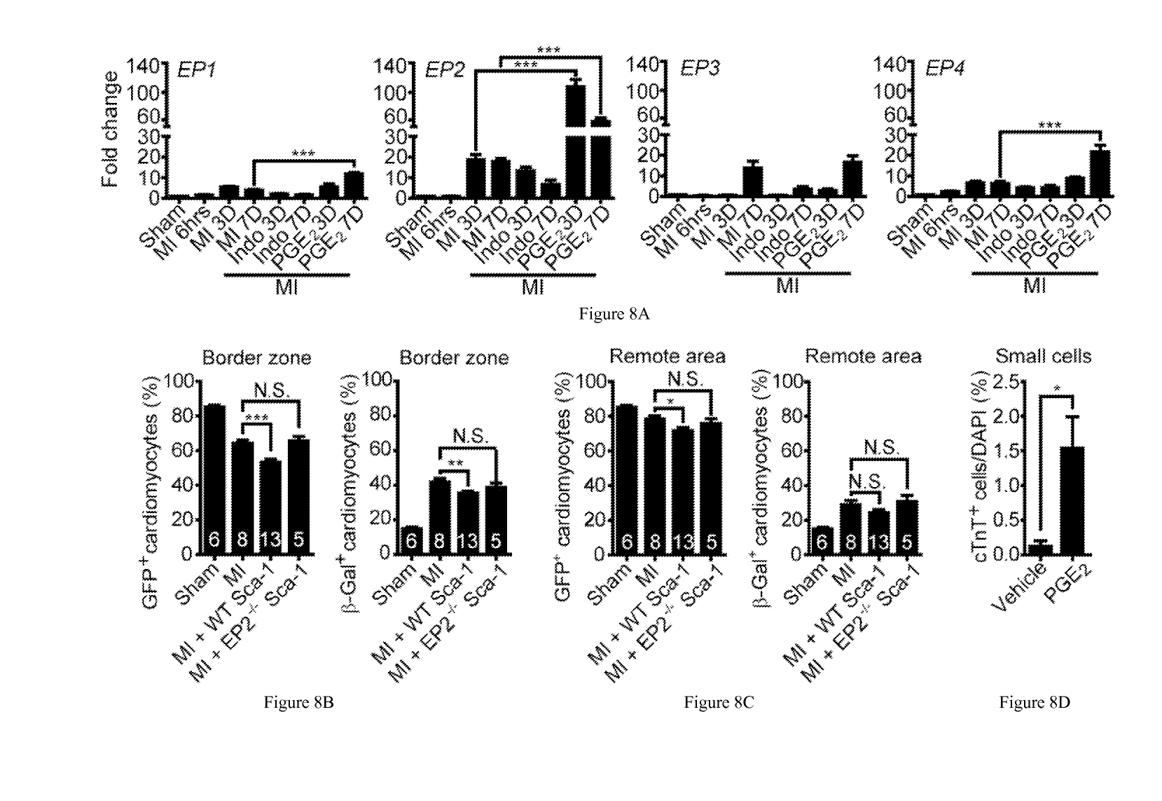

Following MI, M/Z system serves as a platform to assess the cardiomyocytes differentiated from endogenous stem/progenitor cells. Thus, to evaluate the cardiomyocyte differentiation ability of cardiac Sca-1⁺ cells and the importance of PGE2 pathway during this process, cardiac Sca-1⁺ cells were isolated from wild-type and EP2^{-/-} mice (Kennedy, C. R. J. et al. Nat. Med. 5, 217-220 (1999)) for intramyocardial injection after MI surgery. The EP2^{-/-} transgenic mouse was chosen due to expression of this PGE2 receptor was significantly induced in hearts after MI with PGE2 treatment (FIG. 8A). Quantification of the GFP⁺ and β-Gal⁺ cardiomyocyte numbers revealed that injection of wild-type Sca-1⁺ cells reduced both GFP⁺ and β-Gal⁺ cardiomyocyte numbers and that about 10% of the peri-infarct cardiomyocytes were GFP⁻ and β-Gal⁻, thereby suggesting cardiomyocyte differentiation of the injected cardiac Sca-1⁺ cells. In contrast, no such change was observed in the M/Z mice receiving injection of EP2^{-/-} Sca-1⁺ cells (FIGS. 8B and 8C). Together these results indicate that the PGE2/EP2 signaling may regulate the ability of cardiac Sca-1⁺ cells to differentiate into cardiomyocytes. Furthermore, mature sarcomeric structure, as determined by the expression of cTnT, were seen in the cardiomyocyte-depleted small cells (include cardiac Sca-1⁺ cells) after PGE2 treatment (FIG. 8D), thereby suggesting that PGE2 may improve cardiomyocyte differentiation.

### PGE2 Modulates Post-Infarction Inflammatory Response in the Myocardium

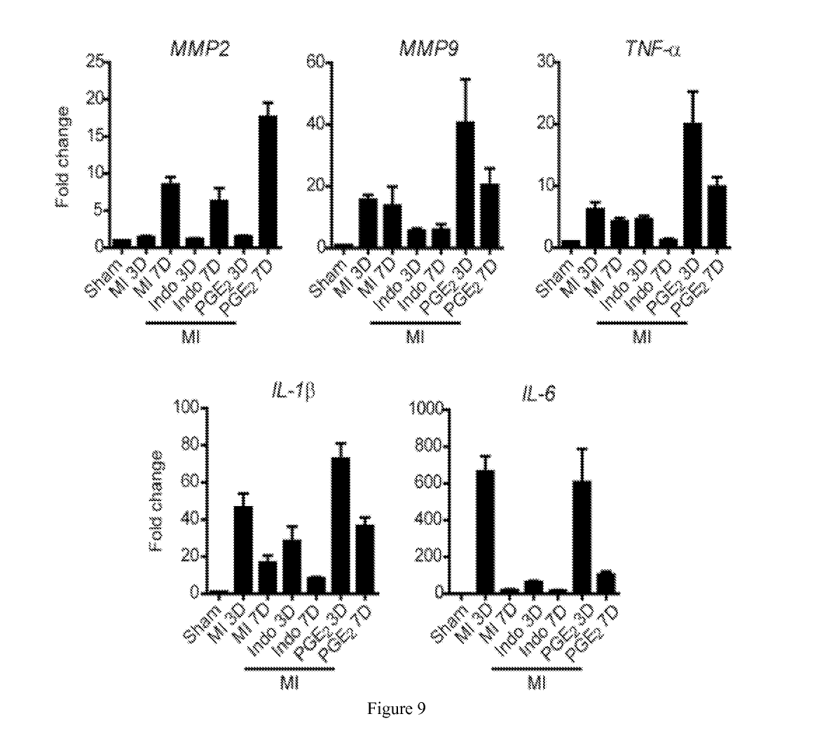

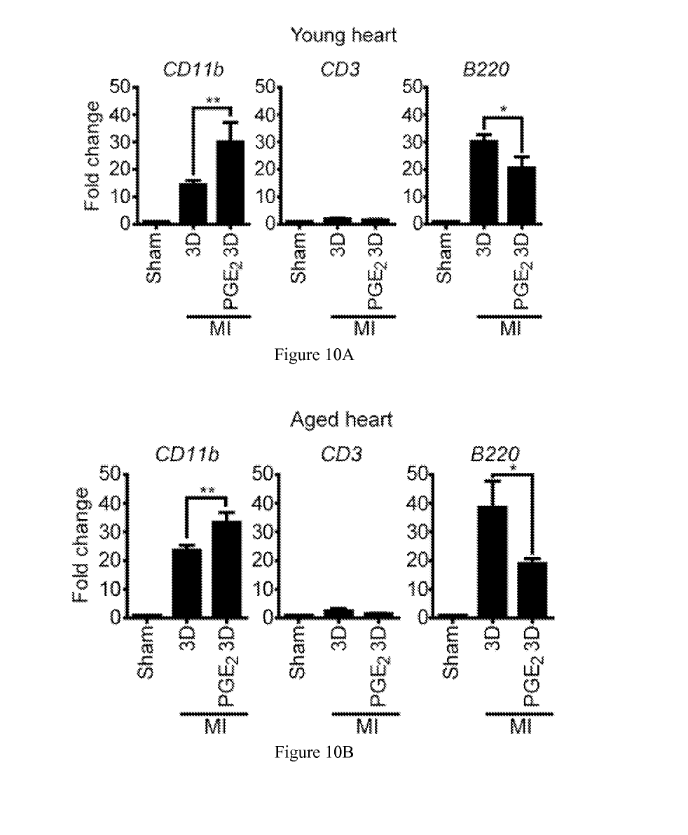

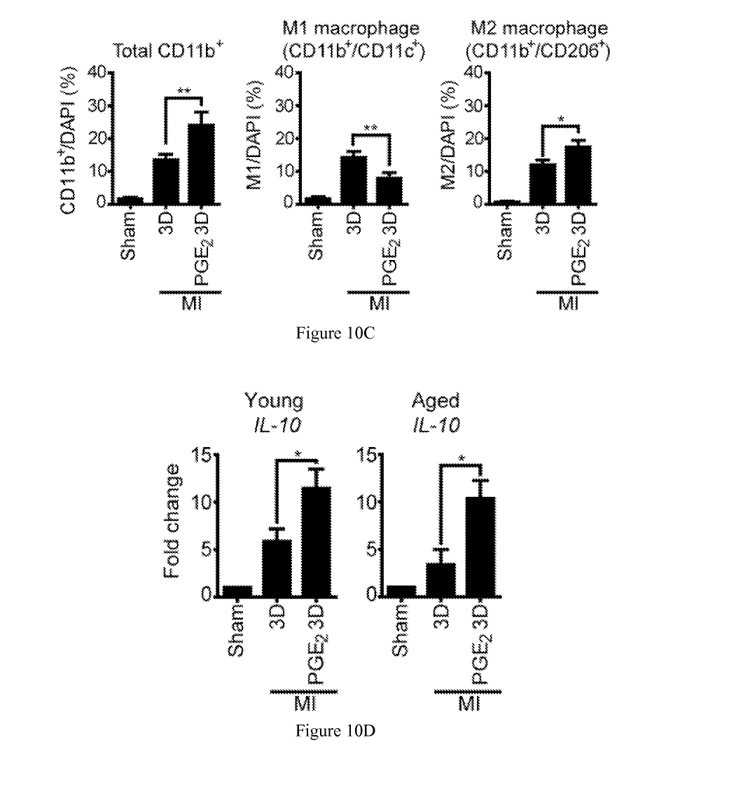

Quantitative RT-PCR analysis revealed that PGE2 treatment further enhanced the expression of inflammatory cytokines acting downstream of PGE2 in the infarct region (FIG. 9). To identify the immune cells underlying PGE2-modulated response, the expression level of inflammatory cell markers, including CD11b for macrophages, B220 for B cells and CD3 for T cells were examined. Following PGE2 administration, CD11b and B220 levels were found to be induced and reduced, respectively, in both young and old mice (FIGS. 10A and 10B). As macrophages can be classified into M1 (CD11b⁺/CD11c⁺) and M2 (CD11b⁺/CD206⁺ or CD68⁺/CD206⁺) subtypes, the effect of PGE2 on the M1 and M2 type macrophages (Nishimura, S. et al. Nat. Med. 15, 914-920 (2009); and Vandanmagsar, B. et al. Nat. Med. 17, 179-188 (2011)) was examined. Immunostaining revealed that PGE2 changed the ratio of M1/M2 macrophages, in which the number of M2 macrophage was increased while the number of M1 macrophage was reduced after PGE2 treatment (FIG. 10C). Furthermore, quantitative RT-PCR indicated that PGE2 enhanced the expression of interleukin-10 (IL-10), which is modulated by M2 macrophages, in both young and old mice (FIG. 10D). Therefore, PGE2 appears the act on not only the progenitor/stem cells directly but also the inflammatory cells such as macrophages to regulate the inflammatory micro-environment after MI.

In summary, the experiments herein show that MI-induced cardiomyocyte replenishment is activated by an early inflammatory response and that this process is saturated within 10 days of injury in mice. The experiments also evidence that PGE2 promotes cardiac differentiation of Sca-1⁺ cells through EP2 receptor, which subsequently contributes to a further increase in cardiomyocyte replenishment. In aged hearts, enhanced TGF-β1 expression may lead to attenuation of regenerative capacity after injury. However, as shown herein, the regenerative capacity after injury in aged hearts can be restored by inhibiting the TGF-β1 signaling pathway and/or binding or activating the EP2 receptors.

Therefore, despite the prior art teachings that endogenous stem and progenitor cells have a negligible role in replenishing lost and/or damaged cardiomyocytes, the present invention provides methods and compositions for inducing, increasing, and/or enhancing the ability of the endogenous stem and progenitor cells to repopulate cardiomyocytes in cardiac tissues.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method for replenishing lost and/or damaged cardiomyocytes in a cardiac tissue in a subject which comprises
inducing, increasing, and/or enhancing endogenous stem cells and/or progenitor cells in the subject to differentiate into new cardiomyocytes by
a) binding and/or activating the prostaglandin E2 receptors; and/or
b) attenuating and/or inhibiting the TGF-β1 signaling pathway;
in the subject.

2. The method of claim 1, wherein attenuating and/or inhibiting the TGF-β1 signaling pathway is by administering at least one TGF-β1 signaling inhibitor such as SB 431542, LY2157299, LDN193189, SB 525334, LY2109761, SB505124, GW788388, Pirfenidone, Y364947, IDT-1, E-616452, and E-616451, preferably IDT-1, E-616452.451, SB 525334, or LY-364947.

3. The method of claim 1, wherein the binding and/or activating the prostaglandin E2 receptors is by administering to the subject an effective amount of a PGE2 compound and/or a PGE2 agonist.

4. The method of claim 3, wherein the PGE2 compound is prostaglandin E2 or a salt thereof, a derivative of a prostaglandin receptor, or a compound having as part of its structural backbone, the following structural formula (I), which may or may not be substituted: 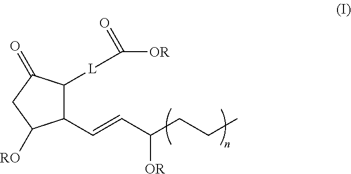
wherein n is an integer between 1 and 10, preferably between 1 and 5, more preferably 2; L is a linker which can be a substituted or unsubstituted alkyl, a substituted or unsubstituted alkenyl; and the R's are each independently H, a halogen, a substituted or unsubstituted C₁₋₆ alkyl, or a substituted or unsubstituted C₁₋₆ alkenyl; and acid, bases, and salts thereof.

5. The method of claim 3, wherein the PGE2 compound and/or the PGE2 agonist is selected from the group consisting of PGE2 and salts thereof, analogues and derivatives of PGE2 and salts thereof, butaprost, CP-533,536, ONO-AE1-259-01, sulprostone, enprostil, and ONO-4819.

6. The method of claim 1, wherein the a) binding and/or activating the prostaglandin E2 receptors; and/or b) attenuating and/or inhibiting the TGF-β1 signaling pathway is conducted before, during and/or after an event likely to cause injury and/or damage to the cardiac tissue.

7. The method of claim 1, wherein the a) binding and/or activating the prostaglandin E2 receptors; and/or b) attenuating and/or inhibiting the TGF-β1 signaling pathway is conducted up to about 3 months, preferably up to about 30 days, more preferably up to about 7-10 days after the event.

8. The method of claim 1, wherein the cardiac tissue is injured or damaged by a myocardial infarction (MI), ischemia, hypoxia, coronary artery disease (CAD), cardiomyopathy, atherosclerosis, heart failure, congenital heart diseases, valvular heart diseases, ischemic heart diseases, and other conditions which damage the cardiac tissue such as viral infection or trauma.

9. The method of claim 8, wherein the cardiovascular disease is heart disease, coronary artery disease, cardiomyopathy, myocardial infarction, atherosclerosis, heart failure, a congenital heart disease, a valvular heart disease, or an ischemic heart disease.

10. The method of claim 3, wherein the effective amount of the PGE2 compound is administered orally, nasally, dermally, mucosally, intravenously, subcutaneously, intramuscularly, or intraperitoneally.

11. The method of claim 4, wherein the effective amount of the PGE2 compound is about 0.001 mg/kg to about 0.1 mg/kg body weight, preferably about 0.001-0.01 mg/kg body weight, or more preferably about 0.0015-0.003 mg/kg body weight, of the subject.

12. The method of claim 5, wherein the effective amount of the PGE2 compound is about 0.001 mg/kg to about 0.1 mg/kg body weight, preferably about 0.001-0.01 mg/kg body weight, or more preferably about 0.0015-0.003 mg/kg body weight, of the subject.

13. The method of claim 1, wherein the effective amount of the PGE2 compound is administered in conjunction with one or more cells to be transplanted to the subject.


SEQUENCE LISTING (48 sequences)

SEQ ID NO: 1 - DNA, 20 bp, Artificial Sequence
  Primer for c-Kit
ctcacatagcagggagcaca

SEQ ID NO: 2 - DNA, 20 bp, Artificial Sequence
  Primer for c-Kit
acaactcacccacacgcata

SEQ ID NO: 3 - DNA, 20 bp, Artificial Sequence
  Primer for Sca-1
acctccacccttgtcctttt

SEQ ID NO: 4 - DNA, 20 bp, Artificial Sequence
  Primer for Sca-1
cttcactgtgctggctgtgt

SEQ ID NO: 5 - DNA, 20 bp, Artificial Sequence
  Primer for Mesp1
ctgaagagtggaggggacaa

SEQ ID NO: 6 - DNA, 20 bp, Artificial Sequence
  Primer for Mesp1
caaggagggttggaatggta

SEQ ID NO: 7 - DNA, 22 bp, Artificial Sequence
  Primer for Flk-1
tctactgggttagcctgtaaac

SEQ ID NO: 8 - DNA, 20 bp, Artificial Sequence
  Primer for Flk-1
tctgtctggctgtcatctgg

SEQ ID NO: 9 - DNA, 23 bp, Artificial Sequence
  Primer for Mef2c
atgccatcagtgaatcaaaggat

SEQ ID NO: 10 - DNA, 19 bp, Artificial Sequence
  Primer for Mef2c
gtggtacggtctcccaact

SEQ ID NO: 11 - DNA, 19 bp, Artificial Sequence
  Primer for Isl-1
cagagtcatccgagtgtgg

SEQ ID NO: 12 - DNA, 22 bp, Artificial Sequence
  Primer for Isl-1
tctactgggttagcctgtaaac

SEQ ID NO: 13 - DNA, 20 bp, Artificial Sequence
  Primer for Nkx2.5
gagcctggtagggaaagagc

SEQ ID NO: 14 - DNA, 20 bp, Artificial Sequence
  Primer for Nkx2.5
tctgagggacagggcatagt

SEQ ID NO: 15 - DNA, 20 bp, Artificial Sequence
  Primer for GATA4
tcaaaccagaaaacggaagc

SEQ ID NO: 16 - DNA, 20 bp, Artificial Sequence
  Primer for GATA4
ctgctgtgcccatagtgaga

SEQ ID NO: 17 - DNA, 21 bp, Artificial Sequence
  Primer for cTnT
cagaggaggccaacgtagaag

SEQ ID NO: 18 - DNA, 23 bp, Artificial Sequence
  Primer for cTnT
tcgatcagagtctgtagctcatt

SEQ ID NO: 19 - DNA, 20 bp, Artificial Sequence
  Primer for MMP2
agcgtgaagtttggaagcat

SEQ ID NO: 20 - DNA, 20 bp, Artificial Sequence
  Primer for MMP2
aggtgaaggagaaggctggt

SEQ ID NO: 21 - DNA, 20 bp, Artificial Sequence
  Primer for MMP9
cgacagcacctcccactatg

SEQ ID NO: 22 - DNA, 20 bp, Artificial Sequence
  Primer for MMP9
ctcagaagagcccgcagtag

SEQ ID NO: 23 - DNA, 20 bp, Artificial Sequence
  Primer for TNF-alpha
gcctcttctcattcctgctt

SEQ ID NO: 24 - DNA, 20 bp, Artificial Sequence
  Primer for TNF-alpha
cacttggtggtttgctacga

SEQ ID NO: 25 - DNA, 20 bp, Artificial Sequence
  Primer for IL-1beta
tgttttcctccttgcctctg

SEQ ID NO: 26 - DNA, 20 bp, Artificial Sequence
  Primer for IL-1beta
tgtgctggtgcttcattcat

SEQ ID NO: 27 - DNA, 20 bp, Artificial Sequence
  Primer for IL-6
tgatgcacttgcagaaaaca

SEQ ID NO: 28 - DNA, 23 bp, Artificial Sequence
  Primer for IL-6
accagaggaaattttcaataggc

SEQ ID NO: 29 - DNA, 20 bp, Artificial Sequence
  Primer for IL-10
agcaaggcagtggagcaggt

SEQ ID NO: 30 - DNA, 22 bp, Artificial Sequence
  Primer for IL-10
ggaactgaggtatcagaggtaa

SEQ ID NO: 31 - DNA, 18 bp, Artificial Sequence
  Primer for TGF-beta 1
cgcaacaacgccatctat

SEQ ID NO: 32 - DNA, 19 bp, Artificial Sequence
  Primer for TGF-beta 1
ccaaggtaacgccaggaat

SEQ ID NO: 33 - DNA, 20 bp, Artificial Sequence
  Primer for CD11b
aaggattcagcaagccagaa

SEQ ID NO: 34 - DNA, 20 bp, Artificial Sequence
  Primer for CD11b
tagcaggaaagatgggatgg

SEQ ID NO: 35 - DNA, 20 bp, Artificial Sequence
  Primer for CD3
ctctcctgcctcctctgcta

SEQ ID NO: 36 - DNA, 20 bp, Artificial Sequence
  Primer for CD3
ggagaggaaaggaactgagg

SEQ ID NO: 37 - DNA, 20 bp, Artificial Sequence
  Primer for B220
gggttgttctgtgccttgtt

SEQ ID NO: 38 - DNA, 20 bp, Artificial Sequence
  Primer for B220
ggatagatgctggcgatgat

SEQ ID NO: 39 - DNA, 20 bp, Artificial Sequence
  Primer for EP1
cgcagggttcacgcacacga

SEQ ID NO: 40 - DNA, 20 bp, Artificial Sequence
  Primer for EP1
cactgtgccgggaactacgc

SEQ ID NO: 41 - DNA, 22 bp, Artificial Sequence
  Primer for EP2
aggacttcgatggcagaggaga

SEQ ID NO: 42 - DNA, 23 bp, Artificial Sequence
  Primer for EP2
cagccccttacacttctccaatg

SEQ ID NO: 43 - DNA, 21 bp, Artificial Sequence
  Primer for EP3
catgatggtcactggcttcgt

SEQ ID NO: 44 - DNA, 21 bp, Artificial Sequence
  Primer for EP3
gtcaccaccagagccagcaag

SEQ ID NO: 45 - DNA, 19 bp, Artificial Sequence
  Primer for EP4
tgcttctgtgaaccccatc

SEQ ID NO: 46 - DNA, 23 bp, Artificial Sequence
  Primer for EP4
gaggtggtgtctgcttgggtcag

SEQ ID NO: 47 - DNA, 18 bp, Artificial Sequence
  Primer for Gapdh
aacgaccccttcattgac

SEQ ID NO: 48 - DNA, 19 bp, Artificial Sequence
  Primer for Gapdh
tccacgacatactcagcac